(19) United States
(12) Patent Application Publication
(10) Pub. No.: US 2025/0269091 A1
(43) Pub. Date: Aug. 28, 2025

(54) UTILIZATION OF ELECTROPHORETIC DEPOSITION TO COAT MEDICAL IMPLANTS WITH THERAPEUTIC AGENTS IN A STERILE CLINICAL SETTING

(71) Applicant: Rush University Medical Center, Chicago, IL (US)

(72) Inventors: John HAMILTON, Deerfield, IL (US); Markus WIMMER, Chicago, IL (US); Adrienn MARKOVICS, Chicago, IL (US); Greta DELLA FARA, Lombardy (IT)

(73) Assignee: Rush University Medical Center, Chicago, IL (US)

(21) Appl. No.: 18/858,215

(22) PCT Filed: Apr. 18, 2023
(86) PCT No.: PCT/US2023/018967

§ 371 Date: Oct. 18, 2024

Related U.S. Application Data
(60) Provisional application No. 63/332,238, filed on Apr. 18, 2022.

(51) Int. Cl. A61L 27/54; A61L 27/06; C25D 13/02; C25D 13/06; C25D 13/12; C25D 13/20

(52) U.S. Cl. CPC A61L 27/54; A61L 27/06; C25D 13/02; C25D 13/06; C25D 13/12; C25D 13/20; A61L 2300/256; A61L 2300/404; A61L 2300/41; A61L 2300/414; A61L 2400/12; A61L 2420/02

(57) ABSTRACT

Disclosed herein are systems and methods for coating an implant with a therapeutic agent in a sterile clinical setting. The systems and methods disclosed herein can provide for coating implants or other prosthetic components with one or more therapeutic agents and/or other compounds within a sterile environment, such as an operating room, either before, during, or after implantation. The systems and methods disclosed herein can utilize electrophoretic deposition to coat an implant after sterilization in connection with implantation. The systems and methods disclosed herein can be used with a variety of implants and apply a variety of substances thereto while in a sterile clinical setting.

### CROSS-REFERENCE TO RELATED APPLICATIONS

This application claims the benefit of U.S. Provisional Application No. 63/332,238, entitled “Utilization of Electrophoretic Deposition to Coat Medical Implants with Therapeutic Agents in a Sterile Clinical Setting,” filed on Apr. 18, 2022. The entire contents of this application are hereby incorporated by reference herein.

### FIELD

This disclosure relates generally to systems and methods for coating medical implants with therapeutic agents and, more particularly, using electrophoretic deposition in a clinical setting after sterilization.

### BACKGROUND

Surgical procedures play an important role in modern medicine. Many surgical procedures involve the placement of an implant of some kind within the body. For example, primary hip and knee arthroplasty, in which all or a portion of a joint is replaced with a prosthetic component, ranks among the top five most common procedures performed each year across all surgical disciplines in the United States. Despite use of systemic antibiotic prophylaxis, biomaterial associated infections from joint replacement, spine surgery, or fracture fixation range from 0.5%-50% of cases depending on patient and surgical risk factors. Staphylococcus aureus accounts for 20-30% of cases of orthopedic device-related infections. Once bacterial adhesion occurs on the implant surface, bacterial biofilm formation occurs, making these bacteria resistant to the host immune response or antimicrobials.

Biomaterial infections can lead to requirement of implant removal and replacement, long courses of medical treatment, as well as poor surgical outcomes, such as potential amputation or even death. Particularly, periprosthetic joint infection (PJI) is the most common reason for revision and re-revision of implants. Almost 1-2% of patients undergoing knee or hip replacement develop PJI, and this rate can be over 20% in patients with risk factors. Total hospital costs for infection associated with joint replacement can often exceed $100,000. Current prevention and treatment strategies for PJI include prolonged systemic antibiotic administration, which has limitations such as low efficiency, poor bioavailability and distribution, lack of selectivity, potential drug overdose and toxicity in non-target tissues, as well as the spread of antibiotic resistance.

To overcome this issue, localized drug delivery systems, such as the incorporation of antibiotics in bone cement, beads, dissolvable sponges, or other surgical components are commonly used. Additionally, surgical implants can be topically coated with an antibiotic material to prevent development of bacterial biofilm and infection at the implant surface. Conventional methods for coating a surgical implant with an antibiotic include the air dry method, in which an antibiotic solution is applied to the implant and air dried prior to placement within the body. Antibiotic compounds can also be incorporated into the surgical procedure by mixing into bone cement or a hydrogel.

Such coatings, however, can have a number of drawbacks. For example, antibiotic-loaded bone cement can have poor characteristics related to osseointegration, drug release, and biodegradability. Antibiotic-loaded hydrogels can have poor characteristics related to drug loading capacity and can take up significant volume. And topical antibiotic applications can have poor characteristics related to uniform coating of an implant surface and sustained drug release over time.

Another coating technology, electrophoretic deposition (EPD), can offer an efficient way to fabricate coatings due to its cost efficiency and simplicity. During EPD, charged particles are deposited onto a substrate of opposite charge. EPD is traditionally carried out in one of two ways, among others: i) cathodic electrophoretic deposition, in which the particles are positively charged and the deposition occurs on the cathode; and ii) anodic electrophoretic deposition, in which the particles are negatively charged and the deposition occurs at the anode.

One challenge to utilizing EPD with medical implants is that the implants need to be sterilized prior to surgical placement. This sterilization process often utilizes autoclaving techniques or gamma radiation that can destroy the overwhelming majority of any therapeutic surface coating applications. Application of EPD is possible by medical device manufacturers, however, the requirement for sterilization of the implant, which is often performed at the hospital prior to surgery, makes EPD coatings impractical. Further, even after an initial sterilization by the manufacturer or by autoclave in a hospital or other clinical setting, medical implants of various sizes are often provided to a surgeon as options on a sterile surgical tray. If these implants are not selected, they are often autoclaved again at the hospital for future use. This process can be repeated several times, which can destroy a manufacturer-applied coating even if it was applied at the time of manufacture in a sterile manner or had some resistance to an initial sterilization procedure.

In addition, a manufacturer-applied implant coating can impose on the customer various requirements for storing and/or handling the implant in a special manner to maintain the viability of the coating. This can include the above-noted restrictions with regard to on-site sterilization, but also include restrictions on more routine pre-sterilization handling and storage.

Moreover, a manufacturer-applied implant coating will would include only whatever therapeutic agent and/or other compound is applied at the time of manufacture. There would be no flexibility for a surgeon or other user to customize the coating based on the particular circumstances surrounding its use. Any such variety in coatings would instead require a manufacturer to make and distribute each different variety as part of its product catalog.

Still further, a manufacturer-applied implant coating would necessarily be provided on only whatever implant the manufacturer offers. There would be no flexibility for a surgeon or other user to apply a coating to any of a variety of implants that may be available and/or desirable to use in a given procedure.

Accordingly, there is a need for improved systems and methods to coat an implant with a therapeutic agent after sterilization of the implant in a sterile clinical setting to help combat post-implantation infection.

### SUMMARY

Disclosed herein are systems and methods for coating an implant with a therapeutic agent in a sterile clinical setting that address the above-noted and other deficiencies in the field. Generally speaking, the systems and methods disclosed herein provide for coating implants or other prosthetic components with one or more therapeutic agents and/or other compounds within a sterile field, such as an operating room, either before, during, or after implantation. The systems and methods disclosed herein can, generally speaking, utilize electrophoretic deposition to coat an implant after sterilization in connection with implantation. The systems and methods disclosed herein can provide a number of advantages, including the ability to work with a variety of implants and apply a variety of substances thereto while in a sterile clinical setting. This can prevent and/or treat biomaterial infection without imposing on manufacturers or users onerous requirements in connection with manufacture/assembly, handling, storage, sterilization, and use.

In one aspect, a method of coating a material can include applying voltage difference on a surface of the material; creating a first charge on the surface; and attracting deposition of one or more therapeutic agents and biomaterials having a second, opposite charge to the material.

Any of a variety of alternative or additional features can be included and are considered within the scope of the present disclosure. For example, in some embodiments, the first charge can be a negative charge and the second charge can be a positive charge. In other embodiments, the first charge can be a positive charge and the second charge can be a negative charge.

In certain embodiments, the method can include sterilizing the material prior to applying a voltage difference. The method can further include forming nanotubes on the surface of the material prior to applying a voltage difference. In some embodiments, the material can be a titanium alloy and the nanotubes can be titanium oxide.

In certain embodiments, applying a voltage difference on the surface of the material can include passing a current from a power source to an electrode contacting the material. Applying a voltage difference on the surface of the material can, in some embodiments, include passing a current from a power source to a first electrode contacting the material, as well as regulating the voltage difference using a second electrode located remotely from the material.

In some embodiments, the material can be located outside of a surgical site. In other embodiments, the material can be located within an open surgical site. In still other embodiments, the material can be located within a closed surgical site.

In certain embodiments, the one or more therapeutic agents and biomaterials can include gentamicin. In some embodiments, the one or more therapeutic agents and biomaterials can include chitosan.

In another aspect, a method of coating an implant can include sterilizing an implant, placing the sterilized implant proximate to a therapeutic agent, creating an electromagnetic potential difference between the sterilized implant and the therapeutic agent, and forming a coating on the sterilized implant comprising the therapeutic agent.

As with the instruments described above, the methods disclosed herein can include any of a variety of additional or alternative steps that are considered within the scope of the present disclosure. For example, in some embodiments the therapeutic agent can include any of antimicrobials, antibodies, or immunomodulators configured to inhibit and treat infection. In one embodiment, the therapeutic agent can include gentamicin. In other embodiments, the therapeutic agent can include any of growth factors or cell signaling molecules configured to enhance tissue regeneration. In still other embodiments, the therapeutic agent can include anti-inflammatory molecules configured to limit inflammation and tissue destruction. In yet other embodiments, the therapeutic agent can include antimicrobial metal ions.

Certain embodiments of the disclosed method can include placing the sterilized implant proximate to biomaterial. In some embodiments, the biomaterial can include chitosan, which can facilitate sustained release of another therapeutic agent. Biomaterials that enhance the sustained release of other therapeutic agents can also be considered a therapeutic agent and can be coated on the surface of a sterilized implant through electrophoretic deposition at or around the same time of one or more other therapeutic agents. Any combination of such coating steps is considered within the scope of the present disclosure.

In some embodiments, the method can further include conditioning a surface of the implant to receive the therapeutic agent. Conditioning the surface of the implant can include forming one or more recesses in the surface.

In certain embodiments, the electromagnetic potential difference can be created using an electrical power source coupled to a plurality of electrodes. In other embodiments, the electromagnetic potential difference can be created using a magnetic field generator.

In some embodiments, the coating on the sterilized implant can be formed prior to implantation in a patient.

In certain embodiments, placing the sterilized implant proximate to a therapeutic agent can include immersing the implant in a container with a liquid solution of the therapeutic agent.

In some embodiments, the coating on the sterilized implant is formed at an open surgical site during implantation in a patient. In certain embodiments, placing the sterilized implant proximate to a therapeutic agent can include delivering a liquid solution of the therapeutic agent to the open surgical site.

In certain embodiments, the coating on the sterilized implant can be formed at a closed surgical site after implantation in a patient. In some embodiments, placing the sterilized implant proximate to a therapeutic agent can include delivering the therapeutic agent to the closed surgical site via a percutaneous injection.

In some embodiments, after sterilizing the implant the method is performed without compromising the sterility of the implant.

In another aspect, a system for coating an implant can include a sterilized implant, a therapeutic agent, and an electromagnetic potential generator. The electromagnetic potential generator can be configured to create an electromagnetic potential difference between the sterilized implant and the therapeutic agent and form a coating on the sterilized implant comprising the therapeutic agent.

In some embodiments, the electromagnetic potential generator can be an electrical power supply. In certain embodiments, the system can further include a plurality of electrodes configured to couple to the electrical power supply. The plurality of electrodes can also include a clamp configured to couple to the sterilized implant. The plurality of electrodes can further include an elongate needle electrode configured to contact the sterilized implant. The plurality of electrodes can also include a collector electrode configured to contact a patient's skin.

In some embodiments, the electromagnetic potential generator can be a magnetic field generator.

In some embodiments, the system can further include a container configured to receive the sterilized implant and the therapeutic agent. The therapeutic agent can be contained in a liquid solution.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a schematic illustration of one embodiment of a system according to the present disclosure configured to coat an implant with a therapeutic agent prior to implantation;

FIG. 2 is an alternative illustration of the system of FIG. 1 in a sterile surgical environment;

FIG. 3 is a schematic illustration of one embodiment of a system to condition an implant surface for enhanced therapeutic agent loading;

FIG. 4 is a bar graph showing a diameter of titanium oxide nanotubes formed by various anodization methods performed using the system of FIG. 3;

FIG. 5 is a microscopic view of titanium oxide nanotube openings formed on a titanium surface using the system of FIG. 3;

FIG. 6 is another microscopic view of titanium oxide nanotubes extending upward from a titanium surface;

FIG. 7 is a schematic illustration of one embodiment of a system for electrophoretic deposition of a therapeutic agent using a two-electrode configuration;

FIG. 8 is a bar graph showing one embodiment of an amount of therapeutic agent deposited on a surface by various EPD methods compared to an air dry method;

FIG. 9 is a table showing one embodiment of projected values for a mass of coating that can be deposited on an implant using various voltages and times in EPD according to the present disclosure;

FIG. 10 is a microscopic view of one embodiment of a thin coating prod formed by EPD according to the present disclosure;

FIG. 11 is a microscopic view showing the presence of a therapeutic agent within titanium oxide nanotubes of an implant;

FIG. 12 is a table showing results of energy-dispersive X-ray spectroscopy (EDS) confirming the presence of a therapeutic agent within titanium oxide nanotubes of an implant;

FIG. 13 is a line graph showing one expression of an amount of a therapeutic agent released from an implant surface over time;

FIG. 14 is a line graph showing another expression of an amount of a therapeutic agent released from an implant surface over time;

FIG. 15 is a line graph showing another expression of an amount of a therapeutic agent released from an implant surface over time;

FIG. 16 is a line graph showing another expression of amount of a therapeutic agent released from an implant surface over time;

FIG. 17 is a depiction of an agar diffusion assay comparing components loaded with a therapeutic agent and a biomaterial;

FIG. 18 is another depiction of an agar diffusion assay comparing components loaded with a therapeutic agent and a biomaterial;

FIG. 19 is a bar graph showing one embodiment of bacteria growth in the presence of a therapeutic agent coating according to the present disclosure;

FIG. 20 is a bar graph showing one embodiment of osteoblast viability after a 24-hour culture with various coated and uncoated surfaces;

FIG. 21 is a schematic illustration of another embodiment of a system according to the present disclosure configured to coat an implant with a therapeutic agent after implantation via an open surgical site;

FIG. 22 is a schematic illustration of another embodiment of a system according to the present disclosure configured to coat an implant with a therapeutic agent after implantation via a closed surgical site;

FIG. 23A is a schematic illustration of one embodiment of a total knee arthroplasty implant disposed in a surgical site with biofilm formation;

FIG. 23B is a detail view of a portion of FIG. 23A;

FIG. 23C illustrates the detail view of FIG. 23B while a needle or probe is percutaneously inserted through the skin;

FIG. 23D illustrates the detail view of FIG. 23B while the needle or probe contacts an implant surface to create a circuit;

FIG. 23E illustrates the detail view of FIG. 23B after coating by a therapeutic agent through EPD and release of the therapeutic agent over time;

FIG. 24 is a depiction of X-rays showing one embodiment of various titanium implants at different times following implantation;

FIG. 25 is a depiction of in vivo bioluminescence imaging for bioluminescent Xen 36. S. aureus bacteria in a mouse model of periprosthetic joint infection with the various titanium implants of FIG. 24 at different times following implantation showing prevalence of bacterial infection;

FIG. 26 is a line graph showing in vivo bioluminescence imaging for bioluminescent Xen 36. S. aureus bacteria in a mouse model of periprosthetic joint infection with the various titanium implants of FIG. 24 at different times following implantation showing prevalence of bacterial infection;

FIG. 27 is a bar graph showing one result of a colony-forming unit analysis associated with the various titanium implants of FIG. 24 showing prevalence of bacterial infection;

FIG. 28 is a bar graph showing another result of a colony-forming unit analysis associated with the various titanium implants of FIG. 24 showing prevalence of bacterial infection; and

FIG. 29 is a bar graph showing another result of a colony-forming unit analysis associated with the various titanium implants of FIG. 24 showing prevalence of bacterial infection.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure. Additionally, to the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices may be used, the size and shape of objects with which the devices may be used, and the methods and procedures in which the devices may be used.

Disclosed herein are systems and methods for coating an implant with a therapeutic agent in a sterile clinical setting. Generally speaking, the systems and methods disclosed herein provide for coating implants or other prosthetic components with one or more therapeutic agents and/or other compounds within a sterile field, such as an operating room, either before, during, or after implantation. The systems and methods disclosed herein can, generally speaking, utilize electrophoretic deposition to coat an implant after sterilization in connection with implantation. The systems and methods disclosed herein can provide a number of advantages, including the ability to work with a variety of implants and apply a variety of substances thereto while in a sterile clinical setting. This can prevent and/or treat biomaterial infection without imposing on manufacturers or users onerous requirements in connection with manufacture/assembly, handling, storage, sterilization, and use.

More particularly, described herein is the ability to use electrophoretic deposition (EPD) to coat medical implant material, including titanium implants, with a therapeutic agent, such as gentamicin antibiotic, and other biomaterial, such as slow-release agent chitosan. In certain embodiments nanotubes or other surface conditioning at the implant surface can be performed prior to EPD to enhance loading of the titanium surface with gentamicin antibiotic. To perform EPD, a negative charge on the titanium surface was created through the application of a voltage difference to attract deposition of gentamicin and chitosan due to their positive charge in solution. Using EPD, a coating was formed containing more than 10× the amount of antibiotics at the titanium surface as compared to application of antibiotics in solution to the implant and drying of solution on the implant (i.e., an air dry method). Gentamicin coating with EPD killed and neutralized bacterial infection with Staphyloccocus aureus and resulted in non-toxicity in osteoblast cultures. Furthermore, dual coating with gentamicin and chitosan enhanced sustained delivery of gentamicin from the implant surface over three days. In other embodiments, medical implants can be coated with antibodies targeted against bacterial biofilm using EPD to enhance prevention and potentially eradicate implant associated infection, something that has not been tried before.

Moreover, EPD can be applied to coating medical implants for substances including, but not limited to: i) antimicrobials, antibodies, or immunomodulators for inhibition and treatment of infection; ii) growth factors or cell signaling molecules to enhance tissue regeneration around the implant; iii) anti-inflammatory molecules to limit inflammation and tissue destruction around the implant; iv) metal ions as an antimicrobial or enhancer of tissue regeneration; v) biomaterials or sustained release agents, such as chitosan, poly(lactic-co-glycolic acid) (PLGA), poly(D,L-lactide), etc., that can enhance sustained release of other therapeutics. In particular, the present disclosure contemplates utilizing EPD to coat an implant with a material that can enhance the coating of another material. For example the present disclosure contemplates systems and methods for coating an implant with a first therapeutic agent, such as an antiobiotic, etc., and a second therapeutic agent and/or other biomaterial, such as chitosan. etc., that can enhance the sustained release of the first therapeutic agent. Such sustained release agents or controlled release agents can increase the effectiveness of the first therapeutic agent coating.

Described herein are applications where EPD could be performed by the medical and/or surgical team that would be performed after implant sterilization, such as, but not limit to, during the surgical procedure in the operating room. The EPD applications disclosed herein can be easy and rapid to perform by the surgical and/or clinical team. For example, EPD can be rapidly performed in approximately 10 minutes or less by a single individual. EPD can therefore be effectively applied to coat implants from a manufacturer in minutes directly prior to surgery.

EPD technology as disclosed herein can be utilized by hospitals and hospital staff in a similar manner as electrosurgery and electrocauterization technologies used routinely today. Such devices have non-sterile components, such as the power source, as well as sterile and disposable components, such as electrodes, that can be utilized in the operating room and utilized to introduce charge/current into the patient.

In addition, EPD technology as disclosed herein poses no novel challenge to hospitals and hospital staff, as they routinely fabricate materials with therapeutic agents in the sterile operating room. For instance, the surgical team is often employed in fabricating bone cement for implants from starting components during surgery and mixing this fabricated bone cement with antibiotics. EPD coating of implants can be faster and easier than preparing and mixing antibiotic loaded bone cement, and can be utilized in the sterile clinical setting by the medical team to coat medical implants with therapeutic agents.

A number of different embodiments for systems and methods of coating implants are disclosed herein and shown in the figures. An overview of several embodiments is provided below:

In a first embodiment, implants can be coated by EPD with electrodes prior to surgical placement by the medical team. In brief, a power source would connect to the implant and other electrodes to allow a charge on the implant surface. This surface charge would allow for electrophoretic deposition of oppositely charged material on the implant surface. In contrast to industrial or laboratory use of a potentiostat or galvanostat configuration, a majority of the instruments in this set-up would be sterile, pre-packed, and can be designed to be disposable allowing for use in a sterile surgical operating environment for each individual implant and patient.

Non-sterile and re-usable instruments (non-disposable) can include the power source, which can have ports that allow for output and input to electrodes. This power source can be re-usable and allow ports for sterile instruments, as discussed below.

Pre-packaged and sterile components that can be disposable can include an implant connector to the power source, e.g., a sterile pre-packed clamp and wire connecting to the power source to allow the implant to serve as the anode or cathode. Another component can be a secondary electrode connected to a wire that connects to the power source. This electrode can serve as either an anode or cathode. The electrode and wire can be sterile and pre-packaged. The system can also include a container that houses an aqueous solution and the implant and electrodes can be provided in a sterile pre-packaged container. The system can further include a bioactive material and solution, which can be provided in either powder or aqueous solution form and can be provided in a sterile pre-packed container. If powder is used, sterile saline or other solution can be used to dissolve the bioactive material. This solution can be placed in the sterile container housing the solution as well as the implant and electrodes.

In another embodiment, implants can be coated by EPD using a magnetic field prior to surgical placement by the medical team. In brief, an applied magnetic field can facilitate development of charge and current on the implant surface. This surface charge can allow for electrophoretic deposition of oppositely charged material on the implant surface. A majority of the instruments in this set-up can be sterile, pre-packed, and can be designed to be disposable allowing for use in a sterile surgical operating environment for each individual implant and patient. Non-sterile and re-usable components can include a magnetic field generator, which can be non-sterile and re-usable in some embodiments. In other embodiments, however, the magnetic field generator can be placed in solution and be sterile.

Pre-packaged and sterile components that can be disposable include a container that houses the aqueous solution and implant. In some embodiments, the magnetic field generator can surround the sterile container. Also provided can be bioactive material and solution provided in either powder or aqueous solution form and packaged in a sterile pre-packed container. If powder is used, sterile saline or other solution can be used to dissolve the bioactive material. This solution can be placed in the sterile container housing the solution as well as implant.

In another embodiment, implants can be coated by EPD at an open surgical site with electrodes by the medical team. Once an implant is surgically placed, it can still be possible to coat the implant by electrophoretic deposition in an open surgical site. In this instance, a current can be placed to the implant, creating an accumulation of charge on the implant surface. This charge accumulation can allow for deposition of oppositely charged bioactive substances on the implant surface at the open operating site. A majority of the components in this set-up can be sterile, pre-packed, and can be designed to be disposable allowing for use in a sterile surgical operating environment for each individual implant and patient.

Non-sterile and re-usable components can include a power source having ports that allow for output and input to electrodes. This power source can be re-usable and allow ports for sterile components, as discussed below.

Pre-packaged and sterile instruments that can be disposable include an active electrode, such as a sterile pre-packed probe/needle electrode and wire to connect to the implant. At the surgical site, the probe can be placed on the implant to distribute charge to the implant. This probe/needle can be either a bipolar or monopolar electrode. Also included can be a dispersive/return electrode, which can be connected to a wire that connects to the power source. For monopolar active electrode use, for example, the return electrode can attach to the patient and collect the current distributed through the patient.

Another component can be bioactive material and solution provided in either powder or aqueous solution form in a sterile pre-packed container. Powder or aqueous form of bioactive material can be placed at the open surgical site around the implant.

In another embodiment, coating of implanted material by EPD can be performed at the open surgical site with a magnetic field by the medical team. Once an implant is placed, it can still be possible to coat the implant by EPD in an open surgical site. In this instance, a magnetic field can be applied to the implant, creating an accumulation of charge on the implant surface. This charge accumulation can allow for deposition of oppositely charged bioactive substances on the implant surface at the open operating site. A majority of the components in this set-up can be sterile, pre-packed, and can be designed to be disposable, allowing for use in a sterile surgical operating environment for each individual implant and patient.

Non-sterile and re-usable components can include the magnetic field generator. Alternatively, the magnetic field generator can be sterilized depending on proximity to the open surgical site.

Pre-packaged and sterile components that can be disposable include bioactive material and solution provided in either powder or aqueous solution form in a sterile pre-packed container. Powder or aqueous form of bioactive material can be placed at the open surgical site around the implant.

In another embodiment, coating of implanted material by EPD at a previously closed surgical site with electrodes can be performed by the medical team. Once an implant is placed, it can still be possible to coat the implant by EPD at the closed surgical site by providing a current/charge to the implant surface at the closed surgical site. In this instance, current can be introduced through a wire and electrode that penetrates the skin. A current can be placed to the implant, creating an accumulation of charge on the implant surface. This charge accumulation can allow for deposition of oppositely charged bioactive substances on the implant surface at the open operating site. A majority of the components in this set-up can be sterile, pre-packed, and can be designed to be disposable, allowing for use in a sterile surgical operating environment for each individual implant and patient.

Non-sterile and re-usable instruments can include a power source having ports that allow for output and input to electrodes. This power source can be re-usable and allow ports for sterile components.

Pre-packaged and sterile instruments that can be disposable include an active electrode, which can be a sterile pre-packed disposable probe/needle electrode, along with a wire to connect to the power source. Through penetrating the skin, the probe can be placed on the implant to distribute charge to the implant. This probe/needle can be either a bipolar or monopolar electrode. Another component can be a dispersive/return electrode connected to a wire that connects to the power source. With monopolar active electrode use, for example, the return electrode can attach to the patient and collect the current distributed through the patient. Another component can be a bioactive material and solution provided in aqueous solution form in a sterile pre-packed container. The aqueous form of bioactive material can be injected around or at the closed surgical implant site, such as an infected knee joint/knee joint replacement implant site.

In still another embodiment, coating of implanted material by EPD at a previously closed surgical site can be performed with a magnetic field by the medical team. Once an implant is placed, it can still be possible to coat the implant by EPD in a previously-closed surgical site. In this instance, a magnetic field can be placed to the implant, creating an accumulation of charge on the implant surface. This charge accumulation can allow for deposition of oppositely charged bioactive substances on the implant surface at the closed operating site.

Non-sterile and re-usable components can include the magnetic field generator. Pre-packaged and sterile instruments that can be disposable include bioactive material and solution provided in either powder or aqueous solution form and in a sterile pre-packed container. Powder or aqueous form of bioactive material can be placed at the closed surgical site around the implant using, e.g., an injection.

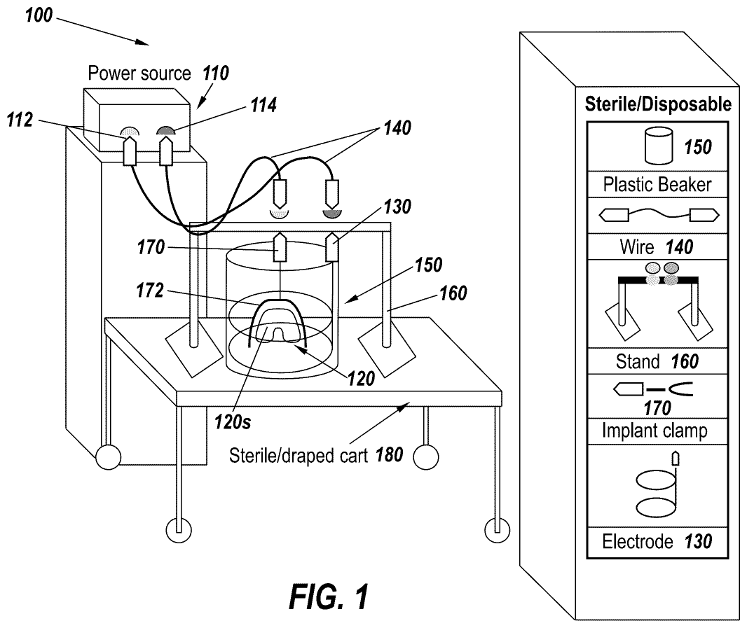

Additional information on various embodiments are provided below. FIG. 1 illustrates a schematic of one embodiment of a surgical system 100 for performing electrophoretic deposition (EPD) to coat medical implants with a therapeutic agent and/or other biomaterial prior to surgical placement by a medical team. The system can include non-sterile or re-usable instruments, such as a power source 110, to supply power to charge the implant surface 120s. The power source 110 can be a potentiostat, galvanostat, or other device for generating electrical voltage and current. The power source 110 can include ports 112, 114 configured to connect to an implant clamp or electrode 170 and a second electrode 130. The electrodes can be coupled to the power source by wire 140 or other material suitable for conducting current. A majority of instruments used in the procedure can be sterilized or disposable in contrast to many industrial or laboratory applications of EPD. Such sterilized or disposable components can include the previously mentioned electrodes 130, 170 and wire 140, as well as, a container 150, support 160, and the implant 120 receiving the coating. In a surgical environment, a user can coat the implant 120 by charging the surface 120s through a current created by the power source 110, implant clamp 170, and electrode 130. In the illustrated two-electrode configuration connected to a potentiostat/power supply, the antibiotic can be provided in solution and the metal implant can be the cathode that attracts the positively charged antibiotics. Adequate antibiotic deposition on the implant surface 120s can occur in approximately 10-15 minutes using a voltage between −4V and −10V.

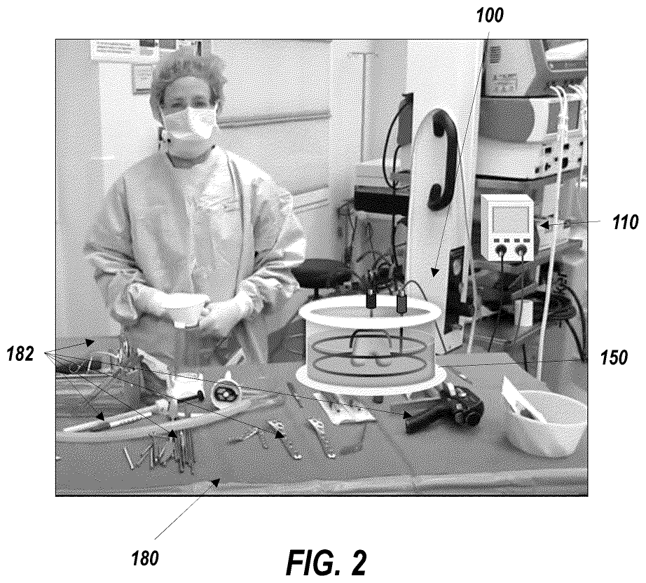

Returning to reference the assembly 100 of FIG. 1, it can be sized such that the EPD method can be performed on a standard sterile cart or preparation table 180 in a sterile operating room, as shown in FIG. 2. The power source 110 and container 150 are shown among various other tools 182 common to surgical preparation as reference to illustrate the size of one embodiment of an EPD system 100 according to the present disclosure.

In the present embodiment, EPD is used to coat a metal femoral component 120 for total knee arthroplasty, although the method can be carried out on various implants made of a medical grade titanium alloy, such as Ti-6Al-4V ELI (extra-low interstitial). Titanium alloy implants can be coated with gentamicin in some embodiments, which is an aminoglycoside antibiotic used to prevent or treat a wide variety of bacterial infections by exerting a concentration-dependent bactericidal effect against microbes associated with infection. Low molecular weight chitosan with sodium tripolyphosphate crosslinking can also be used in order to establish extended release of gentamicin from the implant surface. Chitosan is a naturally occurring, linear polysaccharide used in biomedical applications due to its biodegradability, non-toxicity and versatility. It is a cationic polymer with biocompatible, biodegradable, non-toxic, antibacterial and osteoconductive properties. When used with gentamicin, chitosan acts as a slow-release vehicle, releasing gentamicin from the implant for at least three days after coating. Gentamicin and chitosan molecules carry a positive charge when placed in solutions with a pH below their pKa values (pH=6), which makes them well-suited to use with the EPD techniques disclosed herein since they can be attracted to a negatively-charged surface.

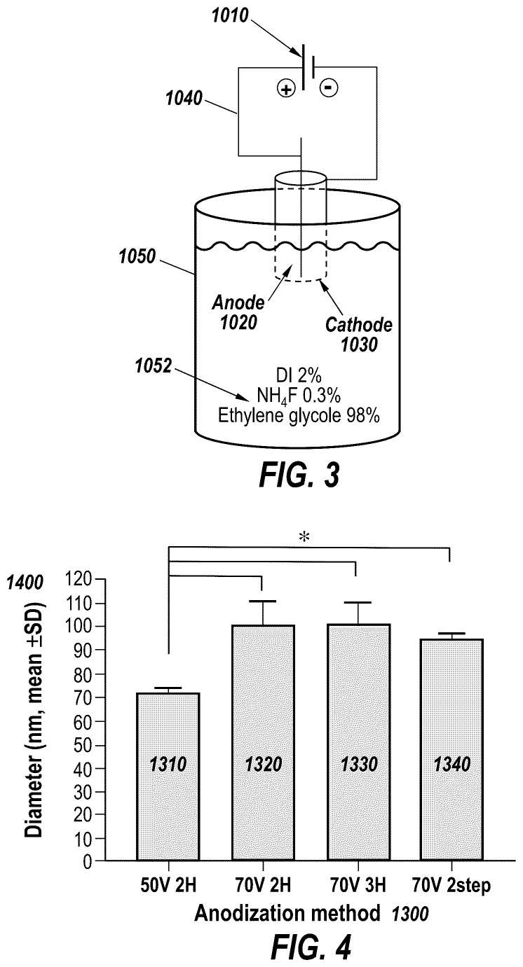

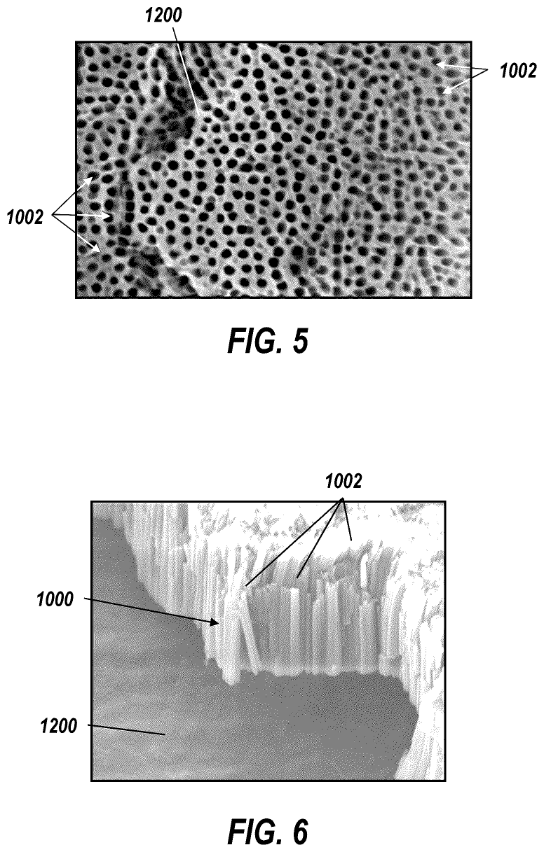

In certain embodiments, it can be beneficial to condition a surface of an implant prior to EPD in order to maximize the ability to coat or load a therapeutic agent onto the implant surface. In some embodiments, conditioning a surface can include forming one or more recesses or voids on the surface that can receive the therapeutic agent during EPD. In one embodiment, Titanium Oxide (TiO₂) nanotubes 1000 (see FIGS. 5 and 6) can be created on the implant surface 1200 by a two-step anodization process to enhance loading of the titanium surface 1200 with an antibiotic or other therapeutic agent. This process can be completed prior to introduction to the sterile environment and prior to sterilization, for example, in a chemical fume hood. Titanium and its alloys are recognized as preferred joint replacement materials due to their excellent biocompatibility, moderate elasticity, and high corrosion resistance. In addition, titanium can spontaneously form a stable and inert layer of titanium oxide on its surface, making it one of the most widely used materials for metallic implants. When the oxide layer is manipulated at the nanoscale, arrays of nanopores, nanopillars, or nanotubes can be created. Nanotubes 1000 are cylindrical hollow structures with diameters ranging from 1-800 nm that can provide an improved level of osseointegration, anti-inflammatory/antimicrobial function, and shielding/scaffolding effects when created on the surface 1200 of an implant. An anodization method for forming TiO₂ nanotubes 1000 on the surface of an implant is illustrated in FIG. 3. The two-electrode configuration comprises a titanium surface 1020, such as the surface of an implant or wire as illustrated, submerged in a solution 1052 and surrounded by a secondary electrode 1030 serving as the cathode. First, an ethylene glycol-based solution 1052 with 0.3 wt % of ammonium-fluoride (NH4F) can be prepared approximately twenty-four hours prior to use to ensure complete NH4F dissolution. In the anodization setup illustrated in FIG. 2, a titanium wire 1020 can be positioned in the middle of a hollow graphite cylinder, which serves as the secondary electrode 1030 or cathode. A power supply 1010 (e.g., Delta Elektronika BV SM700) can be used to provide the required voltage to anodize the surface of the titanium wire 1020. Alternatively, the anodization can be completed in a two-step process comprising of one hour of anodization at 70 V followed by fifteen minutes of sonication in methanol, and an additional thirty minutes of anodization at 70 V. FIG. 4 is a bar chart showing the diameter 1400 of nanotubes formed (nm) by varying anodization methods 1300, such as applying 50V for two hours (1310), applying 70V for two hours (1320), and applying 70V for three hours (1330) compared to the two-step anodization process (1340). The chart demonstrates that higher voltages result in larger titanium oxide nanotube structures during anodization. Top and perspective scanning electron microscopy views of the nanotube structures 1000 are shown in FIGS. 5 and 6. The nanotube creation method disclosed forms amorphous TiO₂ nanotubes 1000 on a microcrystalline Ti6Al4V surface 1200.

As noted above, FIGS. 5 and 6 provide views of the nanotube structures 1000 formed on a titanium surface 1200. In FIG. 5, the openings 1002 of the tubular structures are shown on the surface 1200, while FIG. 6 shows a perspective view of the tubular structures 1000 extending down to the titanium surface 1200. The surface morphology of the nanotubes 1000 shown in the scanning electron microscope views of FIGS. 5 and 6 show highly ordered tubular structures with a 100 nm average diameter at the surface. Based on the morphology obtained after fracture of the coating, the nanotubes were estimated to be between 6 and 7 μm in length. With a diameter of 0.6 mm of the Ti wire shown in FIG. 3 and an 11 mm anodized length, a surface area of about 1700 mm² was obtained, compared to 20 mm² of a non-treated Ti wire.

Referring back to FIG. 1, bioactive material, such as gentamicin and chitosan, can be provided to the sterile environment in either powder or aqueous solution form and stored in a sterile pre-packed container. If powder is used, sterile saline or other solution can be used to dissolve the bioactive material. In the system of FIG. 1, the solution can comprises 30% 50 mg/ml gentamicin and 2 mg/ml chitosan in distilled water and 70% absolute ethanol to prevent water hydrolysis. Solutions with alternative concentrations are also possible in other embodiments. The solution can be placed in the sterile container 150, which can also houses the implant 120 and electrode 130. The sterilized container 150 can be any adequately sized container to hold the implant 120 suspended in an amount of antibiotic-containing solution. The container can be made of plastic or other insulating material such that it does not disrupt the electrochemical process. The container 150 and other components, such as the electrode 130, clamp 170, and wire 140 can be sterilized and pre-packaged separately or as a kit.

To perform the disclosed EPD method in a sterile environment, the sterilized implant 120 can be placed into the container 150 and an antibiotic-containing solution can be added to the container 150 covering the implant 120. A sterilized clamp 170 can be secured to the implant 120 and a wire 140 can connects the clamp 170 to the power source 110. The clamp 170 can suspends the implant 120 in the solution to allow even coating around the entire surface thereof. To facilitate the flow of electric current, the clamp 170 can be made out of a current-conducting material. The electrode 130 can be connected to a wire 140 that connects to the power source 110. A support 160 with current conducting connections can be used to secure the clamp 170 and electrode 130 to one end of wire 140 while the other end of the wire 140 connects back to the power source 110. In the two-electrode configuration, the electrode 130 can serve as either an anode or cathode. Since both gentamicin and chitosan carry a positive charge within the solution, a negative charge on the implant surface is desired to attract the molecules. Therefore, in this system, the electrode 130 serves as an anode and the implant 120 as a cathode within the configuration.

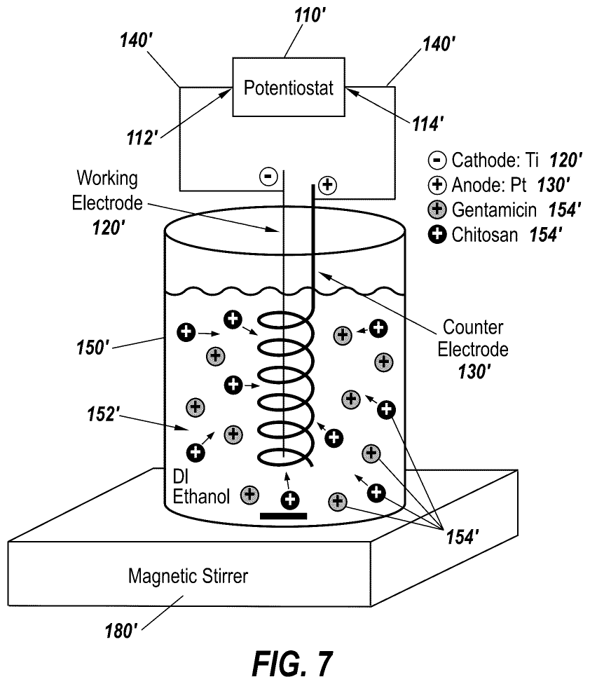

FIG. 7 shows a schematic of the EPD process in further detail. A circuit is created between a power source 110′, implant 120′, and electrode 130′ creating a cathodic potential through the solution 152′ in the sterile container 150′. The secondary or counter electrode 130′ can be connected by wire 140′ to an output port 114′ of a power source 110′ and the implant 120′ can be connected by wire 140′ to an input port 112′ of the power source 110′. The secondary electrode 130′ serving as the anode can be platinum (Pt) in some embodiments. Current passed by the power source 110′ creates a voltage difference between the implant 120′ and secondary electrode 130′ which charges the implant 120′ with a negative charge. The negative charge attracts deposition of the positively charged molecules 154′ within the solution on the implant surface 120′. Adequate antibiotic deposition on the sterile implant surface 120′ can occur in approximately 10-15 minutes using a voltage between −4V and −10V prior to placement within the surgical site. In one embodiment, a voltage of −5 V can be utilized to create a coating of gentamicin and chitosan over about 5 minutes. The EPD method described can be performed in one step or EPD can be performed multiple times with multiple solutions 152′ of varying antibody and carrier compositions and concentrations. For example, gentamicin and chitosan 154′ coatings can be applied as a two-step EPD process. In the first step, a solution 152′ comprising 50 mg/ml gentamicin and 2 mg/ml chitosan can be applied, and in the second step, a 2 mg/ml chitosan solution can be applied. To ensure adequate coating on the entire surface, a magnetic stirrer 180′ and stir bar can be used for thorough mixing of the solution 152′, implant 120′, and electrode 130′ during EPD.

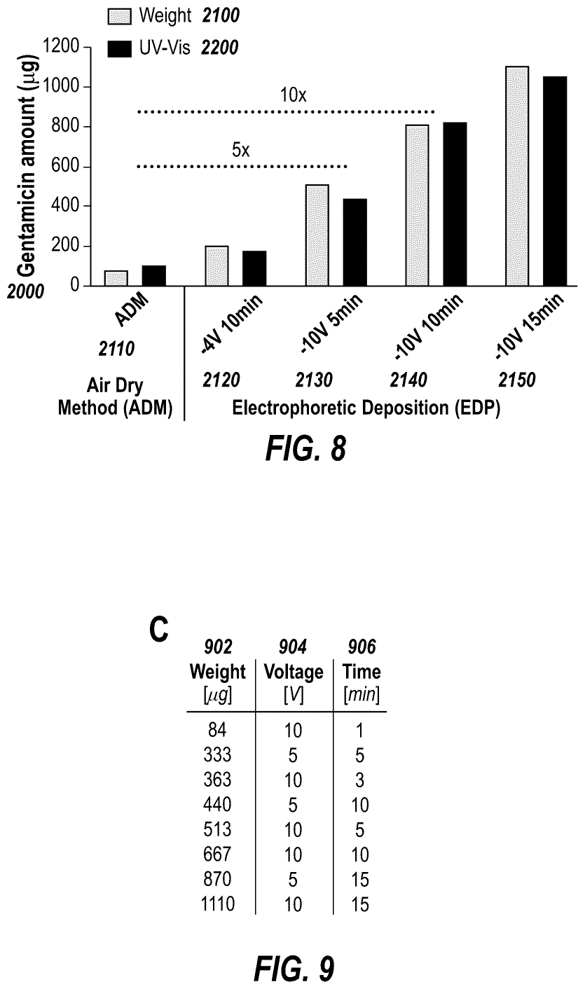

The efficacy of the EPD procedure is shown in the bar chart of FIG. 8. An amount of gentamicin coated on the titanium wire of FIG. 3 was measured by weight 2100 and analysis of a release solution after 10 minutes with UV spectroscopy 2200. The amount of gentamicin (μg) 2000 deposited on a titanium surface with titanium nanotubes by a standard air dry method (ADM) 2110 is compared to the amount deposited by EPD at −4V applied for ten minutes (2120), −10V applied for five minutes (2130), −10V applied for ten minutes (2140), and −10 V applied for fifteen minutes (2150). EPD applied at −10V over five minutes (2130) deposited about five times the amount of gentamicin on the implant surface compared to the standard air dry method 2110, and EPD applied at −10V over ten minutes (2140) deposited ten times the amount of gentamicin on the implant surface as compared to the standard air dry method 2110.

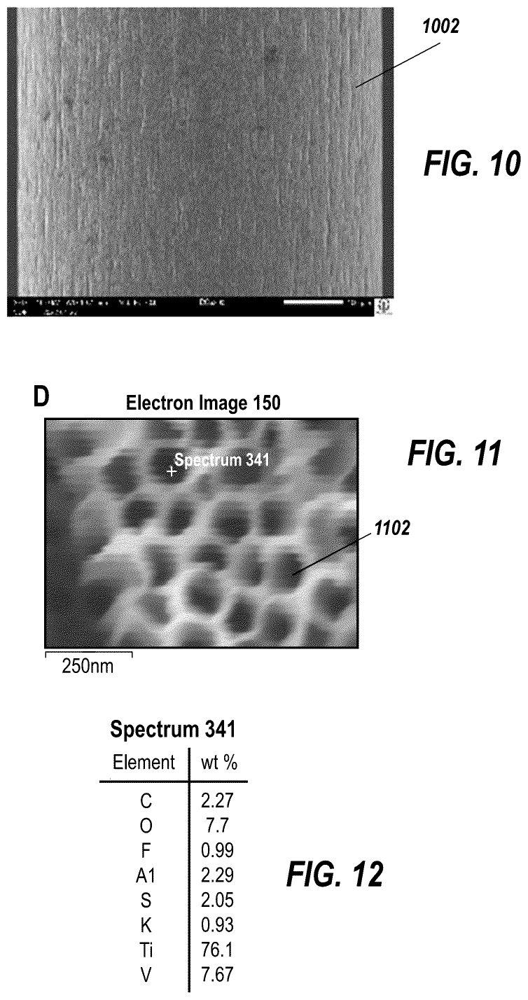

Based on characteristics observed for one embodiment of an EPD process disclosed herein, FIG. 9 shows an amount of drug (by weight 902) loaded onto the titanium rod of FIG. 2 based on voltage (904) applied and duration (906). FIG. 10 shows a scanning electron microscope view of a coating 1002 formed on the titanium rod of FIG. 3 using an EPD process disclosed herein. As shown in FIGS. 11 and 12, scanning electron microscopy in combination with energy-dispersive X-ray spectroscopy was performed at selected pores 1102 in the surface of the titanium rod of FIG. 3. The spectra obtained (see FIG. 12) corresponded to the components of the titanium alloy (Ti, Al, V). In addition, Fluorine (F) was detected, as a remaining product from the anodization process. The presence of Sulfur (S) proved that gentamicin sulfate entered the nanotubes.

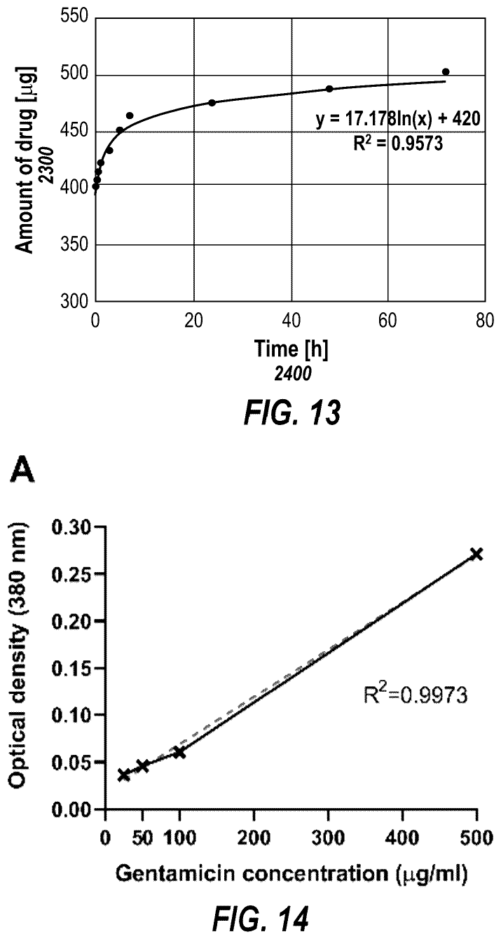

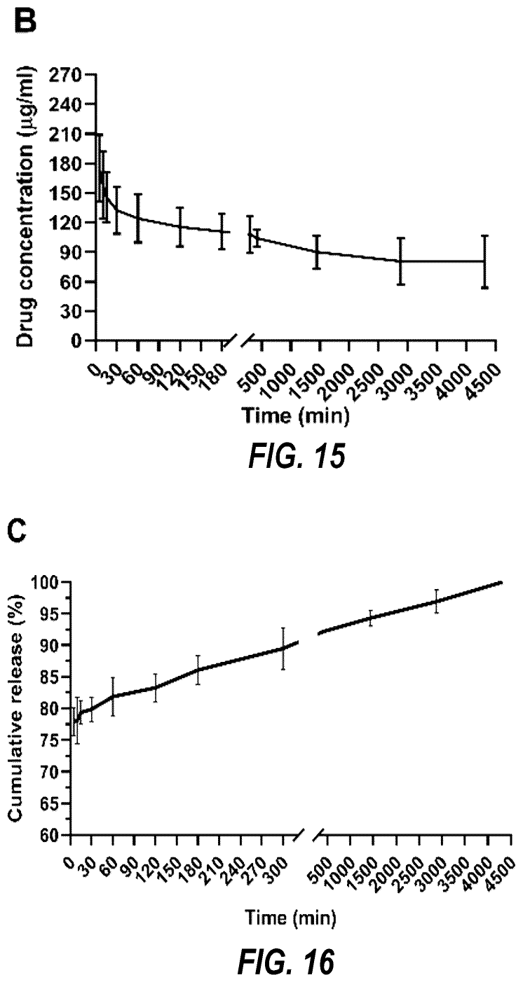

FIG. 13 illustrates a line graph showing the amount of gentamicin 2300 released from a titanium surface per hour 2400 after a two-step EPD process, as described above. FIG. 14 shows a curve resulting from fractional volume sampling to determine an amount of gentamicin released from the titanium nanotubes. Fractional volume sampling was carried out for 3 days. FIG. 15 illustrates the gentamicin concentration in the release solution, and FIG. 16 represents the cumulative release of gentamicin. An initial burst release was observed of around 78% in the first 10 minutes, then gentamicin was released at a slower rate up to 3 days. The drug concentration stayed above the determined minimum inhibitory concentration (MIC) for the entire period tested and a cumulative amount of 400-550 μg gentamicin was detected.

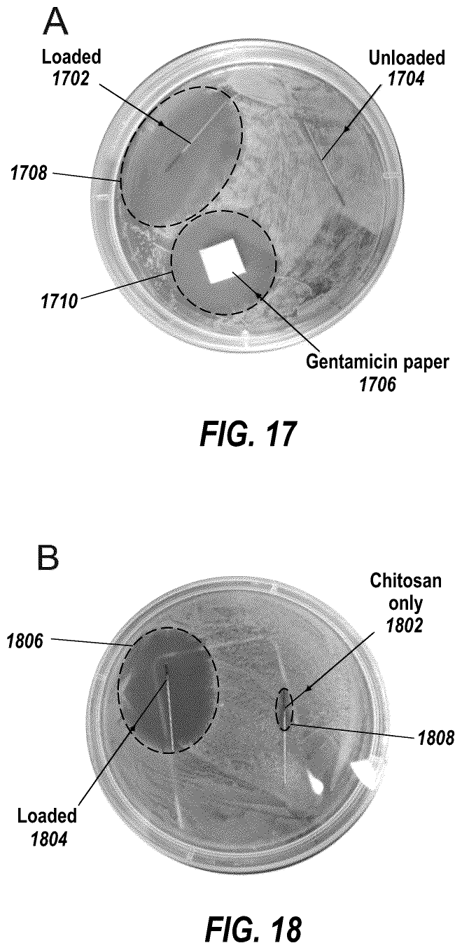

FIGS. 17-20 show tests of the titanium rod of FIG. 3 against Staphylococcus aureus by agar dilution and liquid culture methods. To determine the antimicrobial activity of the loaded wires, S. aureus (Xen36, derived from ATCC 49525) bacteria were cultured overnight at 37° C. in Luria-Bertani medium. The minimal inhibitory concentration of gentamicin was determined by the broth dilution method. In the agar diffusion test, 100 μl of 1×10⁶ colony forming units/ml S. aureus solution was inoculated on agar plates and dried. As shown in FIG. 17, a gentamicin-loaded chitosan-coated wire 1702, an anodized non-loaded wire 1704, and a gentamicin-soaked filter paper 1706 were placed on the plate at a distance. The inhibition zones (IZ) 1708, 1710 around the components were measured following 24 hours incubation at 37° C.

The observed gentamicin MIC was 3.6 μg/ml against S. aureus Xen36. In the agar diffusion assay, an elliptical shaped IZ 1708 formed around the loaded wire 1702 (35 mm shorter and 45 mm longer axis). No IZ was observed around the unloaded wire 1704, thus indicating that the presence of nanotubes alone did not inhibit bacterial growth. The gentamicin-soaked filter paper 1706 resulted in an IZ 1710 of 30 mm in diameter, confirming the effectivity of gentamicin sulfate to inhibit S. aureus growth.

In a separate test shown in FIG. 18, an EPD process with a solution containing chitosan only was performed to investigate if an only chitosan-coated wire 1802 has any antibacterial effect. The elliptical IZ 1806 around the drug-loaded wire 1804 was comparable to the previous one in FIG. 17. A very small IZ 1808 was found around the chitosan-coated wire 1802. This is in accordance with the natural antibacterial effect of chitosan.

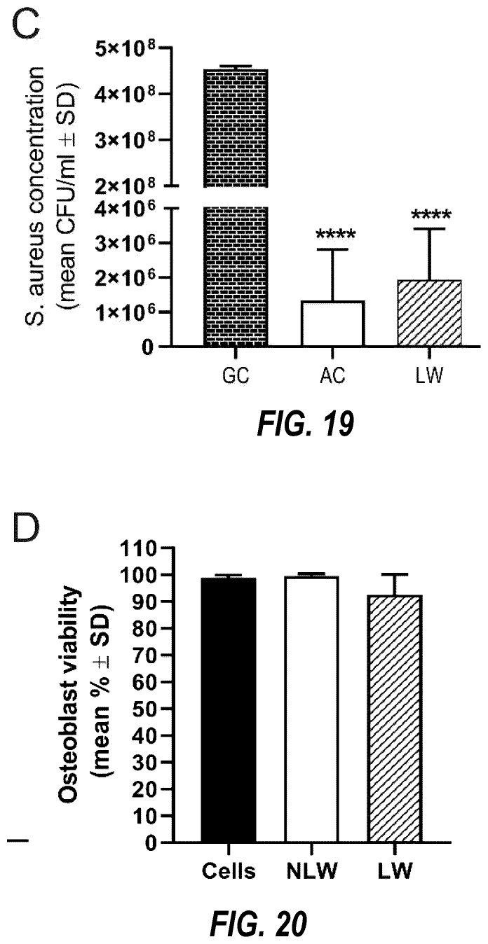

FIG. 19 shows results of a liquid broth culture, in which three tubes with 4 ml of 1×10⁵ CFU/ml S. aureus were prepared. A gentamicin-loaded chitosan-coated wire was placed in one tube (loaded wire, LW). 0.25 mg gentamicin was added to the second tube (antibiotic control, AC). The third tube contained bacteria only and served as growth control (GC). The tubes were incubated for 24 hours, and the absorbance of the solutions was measured with a spectrophotometer in each tube. It was observed that both AC and LW significantly decreased bacterial density compared to GC, further confirming the antibacterial effect of the loaded wire.

Referring to FIG. 20, mouse osteoblast-like cell line (MC3T3-E1) was cultured in Dulbecco's Modified Eagle Medium supplemented with 10% fetal bovine scrum. Titanium wires were placed into the wells of a tissue culture plate. Cells were seeded at 2.5×10⁴ cells/cm² density and cultured (1) without wire (Cells), (2) with anodized, non-loaded wire (NLW), (3) with gentamicin-loaded wire (LW). Cell viability was determined following 24 hours of incubation at 37° C., 5% CO₂, by a Vi-Cell XR cell viability analyzer. The graph of FIG. 20 illustrates the percent viable cells compared to the total number of mouse MC3T3-E1 cells after 24 hours of incubation with anodized, non-loaded wire (NLW), gentamicin-loaded wire (LW) or without wire (Cells). While a lower cell viability was observed in the LW group, the difference was not statistically significant between the groups. Therefore, the gentamicin-loaded chitosan-coated Titanium wires are shown to be effective against S. aureus without significantly affecting the viability of healthy cells.

Other embodiment of systems and methods for EPD coating of implants in a sterile setting are also provided herein. For example, in some embodiments a system and method similar to that described above in connection with FIG. 1 can be utilized, but a magnetic field can be applied to charge the implant surface 120s in place of an electric current. The resulting surface charge can allow for the deposition of oppositely charged antibiotic material on the implant surface 120s, similar to the manner described with respect to FIG. 1. In these embodiments, a magnetic field generator is can be used in place of an electrical power source 110. Similar to the electrical power source 110, the magnetic field generator can be non-sterile and re-usable. Alternatively, the magnetic field generator can be sterilized prior to use and disposed in solution with the implant. That is, the magnetic generator can be placed proximate to the sterilized implant 120 submerged in an antibiotic containing solution in a sterile container 150 and generate a magnetic field through the solution. The magnetic generator can surround the sterile container 150 to generate an even magnetic field through the solution and around the implant 120. The magnetic field can create a negative charge on the implant surface 120s, which can attract the deposition of positively charged molecules on the implant surface 120s.

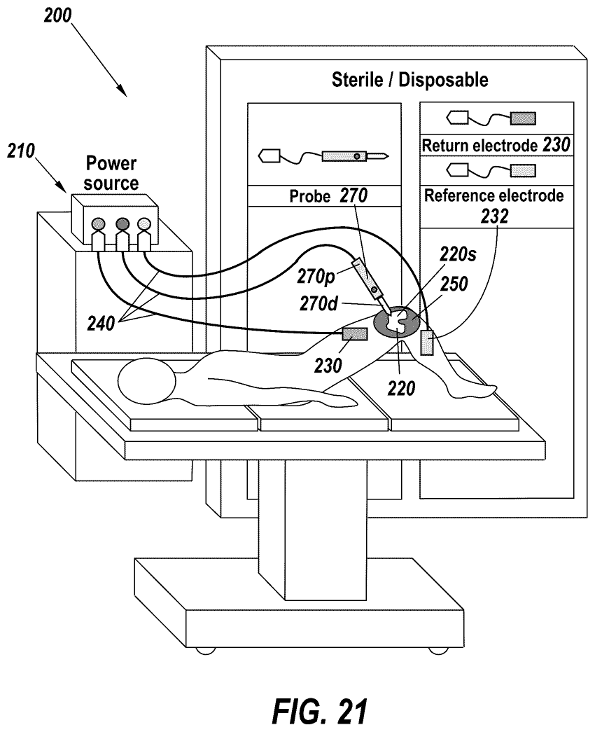

While the methods discussed so far contemplate coating an implant outside of a patient's body prior to insertion in the surgical site, FIG. 21 illustrates a system and method in which an implant 220 can be coated after it is placed in the surgical site 250. Once an implant 220 is surgically positioned in a patient's body, it can still be possible to coat the implant 220 by electrophoretic deposition in an open surgical site. In the system 200 shown in FIG. 21, a current can contact the implant surface 220s, creating an accumulation of charge on the surface 220s. This charge accumulation can allow for deposition of oppositely charged bioactive substances on the implant surface 220s at the open operating site 250. A majority of the components in this set-up can be sterile, pre-packed, and disposable, allowing for use in a sterile surgical operating environment for each individual implant and patient.

In contrast to the system and method illustrated in FIG. 1, the system and method of FIG. 21 can be performed on an implant 220 placed in an open surgical site 250. The system and method illustrated in FIG. 21 can be performed to prevent or treat infection and biofilm formation on the implant surface 220s during primary or revision surgery, such as knee arthroplasty, as well as during debridement, antibiotics, and implant retention (DAIR) procedures, among other procedures. Similar to the embodiment disclosed in FIG. 1, a non-sterile reusable power source 210, such as a potentiostat (e.g., Interface 1010E potentiostat Gamry Instruments) can be used to supply current. The remaining components of the system 200 can include wires 240, a probe 270, return electrode 230, and reference electrode 232, all of which can be sterile and pre-packaged for disposable use. The procedure preferably begins by applying an antibiotic containing solution, such as the solutions previously disclosed, to an implant 220 disposed in the open surgical site 250 such that the solution surrounds the implant 220 and contacts the implant surface 220s. A sterile return electrode 230 can be placed proximate to the implant 220 and open surgical site 250. Sterile wire 240 comprised of current conducting material can connect the return electrode 230 to the potentiostat 210. A probe 270 made of current conducting material can be connected at its proximal end 270p to the potentiostat 210 by wire 240. The distal end of the probe 270d can be configured to contact the implant surface 220s and distribute charge to the surface 220s. The potentiostat 210 can create a voltage difference at the surgical site 250 between the implant 220 and return electrode 230, which can be monitored by a reference electrode 232 disposed on the patient remote from the surgical site 250. The negatively charged implant surface 220s can attract deposition of positively charged molecules within the surrounding solution at the surgical site 250. In this sense, the metal implant can be the cathode and attract the positively charges antibiotics. Adequate antibiotic deposition and coating can occur on the sterile implant surface 220s within an open surgical site by applying a safe voltage between −4V and −10V for approximately 10-15 minutes.

In another embodiment, a similar system and method can be utilized in connection with a magnetic field generator in place of an electric power supply. The magnetic field generator can be used to supply a magnetic field around the implant, facilitating deposition at the implant surface 220s. A magnetic generator can be sterilized and placed proximal to the implant 220 within an open surgical site 250 such that a magnetic field creates an accumulation of negative charge at the implant surface 220s that attracts positively charged molecules in a solution surrounding the implant. Alternatively, the magnetic field generator can be a reusable, non-sterile component effective to create the desired field from a distance without violating the sterile field or environment.

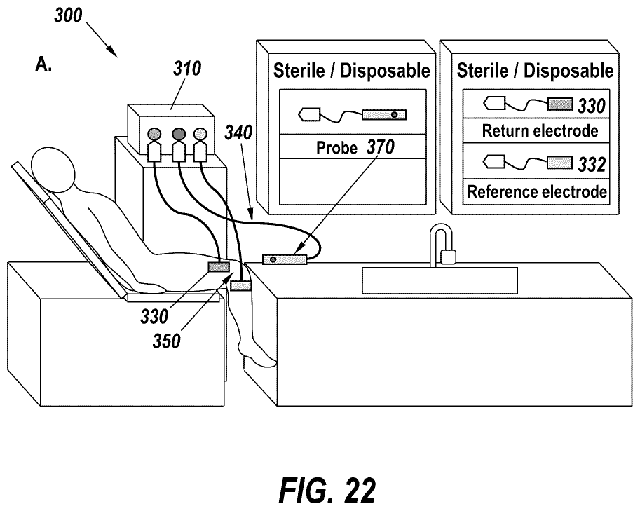

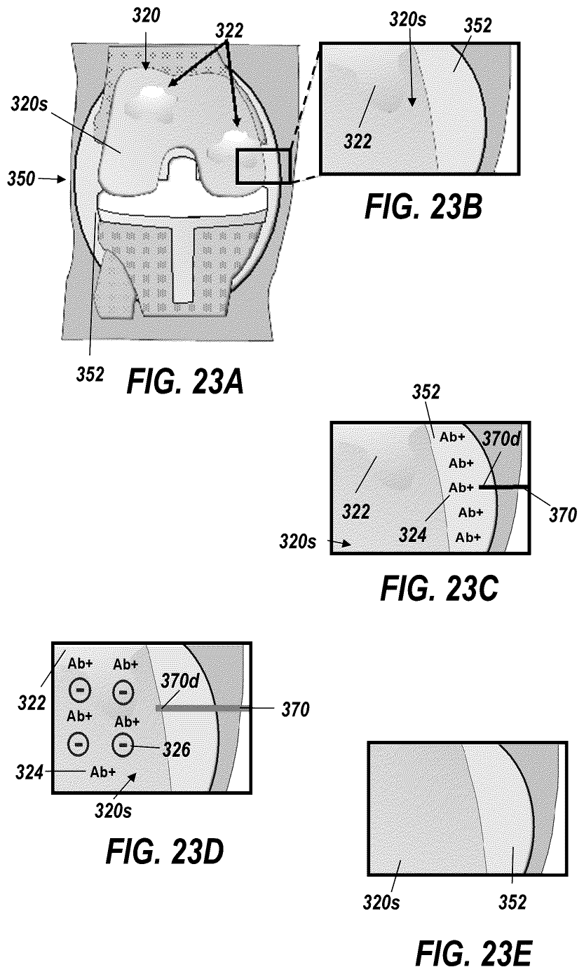

In cases where a bacterial biofilm forms on an implant surface after a surgical site has been closed, the systems and methods disclosed herein can be applied to an implant through the skin in a clinical setting to address infection. An embodiment of such a system and method is illustrated in FIG. 22. In this embodiment, current can be introduced through a wire 340 and probe 370 that penetrates the skin 350 and contacts the implant 320 (see FIG. 23A). The charge created on the surface of implant 320 by the probe 370 at the closed surgical site 350 allows for deposition of oppositely charged bioactive substances injected into the surgical site onto the implant surface 320s (see FIG. 23A). Since the surgical site 350 is closed, the method can be performed in a clinical setting using percutaneously-introduced components. A majority of the components in this set-up can be sterile, pre-packed, and disposable, allowing for use in a clinical environment for each individual implant and patient.

FIG. 23A shows a total knee joint arthroplasty implant 320 in a closed surgical site 350 with bacterial biofilm 322 forming on the surface 320s. An antibiotic containing solution 352, such as those previously disclosed, can be injected percutaneously so it contacts and surrounds the implant 320 in the intra-articular space and synovial fluid, as shown in FIG. 23B. In FIG. 23C, a current conducting needle or probe 370 penetrates through the skin percutaneously until the distal end 370d contacts the implant surface 320s, as shown in FIG. 23D. The proximal end of the probe 370 can be connected to a power source or potentiostat 310 by a wire 340, as shown in FIG. 22. A current is passed from the power source 310 to the probe 370 to the implant 320, creating a voltage difference between the implant 320 and return electrode 330. The voltage difference accumulates a negative charge 326 on the implant surface 320s and positively charged molecules 324 within the surrounding solution 352 are subsequently attracted to the surface 320s, coating the implant 320. Alternatively, a magnetic generator can be placed proximate to the closed surgical site to provide a magnetic field to accumulate a negative charge 326 on the surface of the implant 320s. The antibiotic 324 percutaneously deposited on the implant surface 320s through the disclosed EPD method can fight infection causing bacterial biofilm 322 accumulating on the implant surface 320s within the body as the antibiotic 324 is released from the implant surface 320s over time, as shown in FIG. 23E.

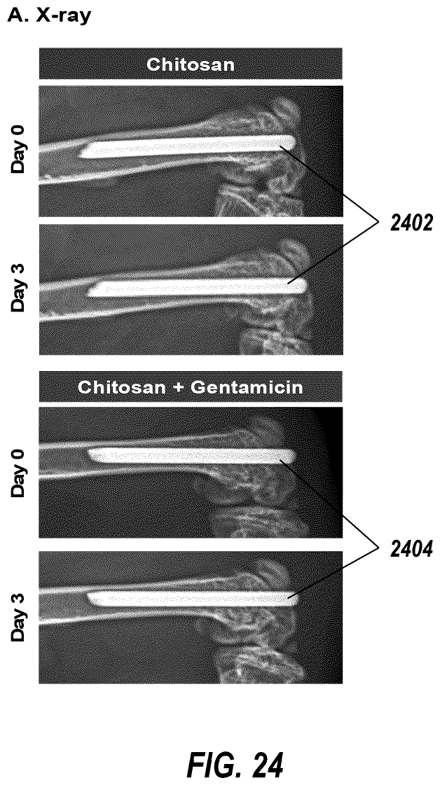

FIGS. 24-29 show an example mouse model of periprosthetic joint infection conducted utilizing implants with titanium nanotubes and electrophoretic deposition of gentamicin and chitosan. In particular, FIG. 24 shows X-rays of titanium femoral nail implants in mice at time of implantation and three days after implantation. The upper images show a titanium implant 2402 having titanium nanotubes formed on a surface thereof and solely chitosan deposited according to the EPD processes described herein. The lower images show a titanium implant 2404 having titanium nanotubes formed on a surface thereof and a coating of gentamicin and chitosan according to the EPD processes described herein.

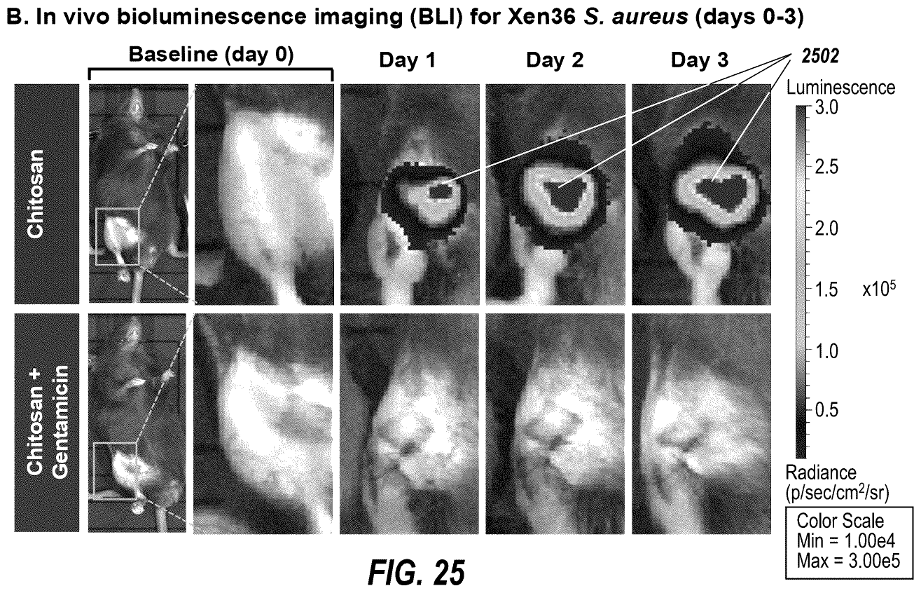

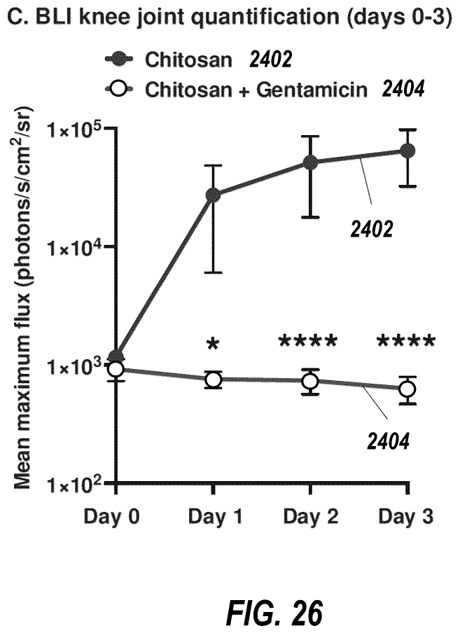

FIGS. 25 and 26 show results of in vivo bioluminescence imaging of Xen36 S. aureus. In the upper images of FIG. 25, images of the area surrounding implant 2402 coated with chitosan only show evidence of infection 2502 developing over the three days following implantation. In the lower images of FIG. 25, images of the area surrounding implant 2404 coated with gentamicin and chitosan show no similar evidence of infection. The line chart of FIG. 26 similarly shows that levels of mean maximum flux measured remain level for the gentamicin-coated implant 2404 and increase for the chitosan-only-coated implant 2402.

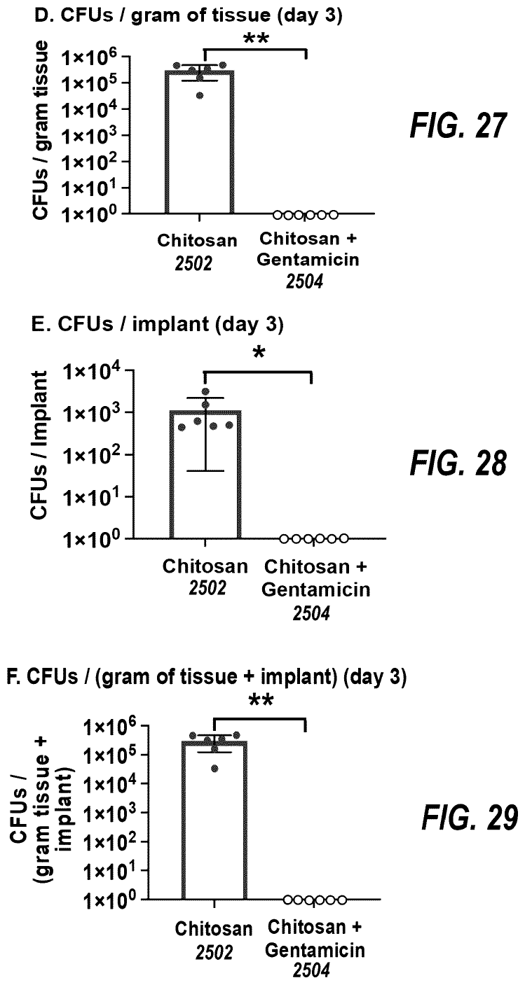

FIGS. 27-29 show results of colony-forming unit (CFU) analysis three days after implantation. These charts all confirm that the implant 2404 coated with gentamicin and chitosan had no evidence of infection, in contrast to the implant 2402 coated with chitosan alone.

Various devices and methods disclosed herein can be used in minimally-invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those which are suitable for use in surgical applications, including metals such as stainless steel, titanium, nickel, cobalt-chromium, or alloys and combinations thereof, polymers such as PEEK, ceramics, carbon fiber, and so forth. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A method of coating a material, comprising:
   applying voltage difference on a surface of the material;
   creating a first charge on the surface; and
   attracting deposition of one or more therapeutic agents and biomaterials having a second, opposite charge to the material.
2. The method of claim 1, wherein the first charge is a negative charge and the second charge is a positive charge.
3. The method of claim 1, wherein the first charge is a positive charge and the second charge is a negative charge.
4. The method of any of claims 1 to 3, further comprising sterilizing the material prior to applying a voltage difference.
5. The method of any of claims 1 to 4, further comprising forming nanotubes on the surface of the material prior to applying a voltage difference.
6. The method of claim 5, wherein the material is a titanium alloy and the nanotubes are titanium oxide.
7. The method of any of claims 1 to 6, wherein applying a voltage difference on the surface of the material further comprises passing a current from a power source to an electrode contacting the material.
8. The method of any of claims 1 to 6, wherein applying a voltage difference on the surface of the material further comprises:
   passing a current from a power source to a first electrode contacting the material; and
   regulating the voltage difference using a second electrode located remotely from the material.
9. The method of any of claims 1 to 8, wherein the material is located outside of a surgical site.
10. The method of any of claims 1 to 8, wherein the material is located within an open surgical site.
11. The method of any of claims 1 to 8, wherein the material is located within a closed surgical site.
12. The method of any of claims 1 to 11, wherein the one or more therapeutic agents and biomaterials comprise gentamicin.
13. The method of any of claims 1 to 12, wherein the one or more therapeutic agents and biomaterials comprise chitosan.
14. A method of coating an implant, comprising:
   sterilizing an implant;
   placing the sterilized implant proximate to a therapeutic agent; and
   creating an electromagnetic potential difference between the sterilized implant and the therapeutic agent;
   forming a coating on the sterilized implant comprising the therapeutic agent.
15. The method of claim 14, wherein the therapeutic agent comprises any of antimicrobials, antibodies, or immunomodulators configured to inhibit and treat infection.
16. The method of claim 15 wherein the therapeutic agent comprises gentamicin.
17. The method of any of claims 14 to 16, wherein the therapeutic agent comprises any of growth factors or cell signaling molecules configured to enhance tissue regeneration.
18. The method of any of claims 14 to 17, wherein the therapeutic agent comprises anti-inflammatory molecules configured to limit inflammation and tissue destruction.
19. The method of any of claims 14 to 18, wherein the therapeutic agent comprises antimicrobial metal ions.
20. The method of any of claims 14 to 19, further comprising placing the sterilized implant proximate to biomaterial.
21. The method of claim 20, wherein the biomaterial comprises chitosan.
22. The method of any of claims 14 to 21, further comprising conditioning a surface of the implant to receive the therapeutic agent.
23. The method of claim 22, wherein conditioning the surface of the implant includes forming one or more recesses in the surface.
24. The method of any of claims 14 to 23, wherein the electromagnetic potential difference is created using an electrical power source coupled to a plurality of electrodes.
25. The method of any of claims 14 to 23, wherein the electromagnetic potential difference is created using a magnetic field generator.
26. The method of any of claims 14 to 25, wherein the coating on the sterilized implant is formed prior to implantation in a patient.
27. The method of claim 26, wherein placing the sterilized implant proximate to a therapeutic agent includes immersing the implant in a container with a liquid solution of the therapeutic agent.
28. The method of any of claims 14 to 25, wherein the coating on the sterilized implant is formed at an open surgical site during implantation in a patient.
29. The method of claim 28, wherein placing the sterilized implant proximate to a therapeutic agent includes delivering a liquid solution of the therapeutic agent to the open surgical site.
30. The method of any of claims 14 to 25, wherein the coating on the sterilized implant is formed at a closed surgical site after implantation in a patient.
31. The method of claim 30, wherein placing the sterilized implant proximate to a therapeutic agent includes delivering the therapeutic agent to the closed surgical site via a percutaneous injection.
32. The method of any of claims 14 to 31, wherein, after sterilizing the implant, the method is performed without compromising the sterility of the implant.
33. A system for coating an implant, comprising:
   a sterilized implant;
   a therapeutic agent; and
   an electromagnetic potential generator;
   wherein the electromagnetic potential generator is configured to create an electromagnetic potential difference between the sterilized implant and the therapeutic agent and form a coating on the sterilized implant comprising the therapeutic agent.
34. The system of claim 33, wherein the therapeutic agent is contained in a liquid solution.
35. The system of any of claims 33 to 34, further comprising a container configured to receive the sterilized implant and the therapeutic agent.
36. The system of any of claims 33 to 35, wherein the electromagnetic potential generator is an electrical power supply.
37. The system of claim 36, further comprising a plurality of electrodes configured to couple to the electrical power supply.
38. The system of claim 37, wherein the plurality of electrodes includes a clamp configured to couple to the sterilized implant.
39. The system of any of claims 37 to 38, wherein the plurality of electrodes includes an elongate needle electrode configured to contact the sterilized implant.
40. The system of any of claims 37 to 39, wherein the plurality of electrodes includes a collector electrode configured to contact a patient's skin.
41. The system of any of claims 33 to 40, wherein the electromagnetic potential generator is a magnetic field generator.

What is claimed is:

1. A method of coating a material, comprising:
applying voltage difference on a surface of the material;
creating a first charge on the surface; and
attracting deposition of one or more therapeutic agents and biomaterials having a second, opposite charge to the material.

2. The method of claim 0, wherein the first charge is a negative charge and the second charge is a positive charge.

3. The method of claim 0, wherein the first charge is a positive charge and the second charge is a negative charge.

4. The method of claim 0, further comprising sterilizing the material or forming nanotubes on the surface of the material prior to applying a voltage difference.

5.-6. (canceled)

7. The method of claim 0, wherein applying a voltage difference on the surface of the material further comprises passing a current from a power source to an electrode contacting the material.

8. (canceled)

9. The method of claim 0, wherein the material is located outside of a surgical site.

10. The method of claim 0, wherein the material is located within an open surgical site.

11. The method of claim 0, wherein the material is located within a closed surgical site.

12. The method of claim 0, wherein the one or more therapeutic agents and biomaterials comprise gentamicin or chitosan.

13. (canceled)

14. A method of coating an implant, comprising:
sterilizing an implant;
placing the sterilized implant proximate to a therapeutic agent; and
creating an electromagnetic potential difference between the sterilized implant and the therapeutic agent;
forming a coating on the sterilized implant comprising the therapeutic agent.

15. The method of claim 14, wherein the therapeutic agent comprises any of antimicrobials, antibodies, or immunomodulators configured to inhibit and treat infection.

16. The method of claim 14, wherein the therapeutic agent comprises any of growth factors or cell signaling molecules configured to enhance tissue regeneration.

17. The method of claim 14, wherein the therapeutic agent comprises anti-inflammatory molecules configured to limit inflammation and tissue destruction.

18. The method of claim 14, wherein the therapeutic agent comprises antimicrobial metal ions.

19.-20. (canceled)

21. The method of claim 14, wherein the electromagnetic potential difference is created using an electrical power source coupled to a plurality of electrodes.

22. The method of claim 14, wherein the electromagnetic potential difference is created using a magnetic field generator.

23. The method of claim 14, wherein the coating on the sterilized implant is formed prior to implantation in a patient.

24. (canceled)

25. The method of claim 14, wherein the coating on the sterilized implant is formed at an open surgical site during implantation in a patient.

26. (canceled)

27. The method of claim 14, wherein the coating on the sterilized implant is formed at a closed surgical site after implantation in a patient.

28. (canceled)

29. The method of claim 14, wherein, after sterilizing the implant, the method is performed without compromising the sterility of the implant.
